(19) United States
(12) Plant Patent Application Publication
(10) Pub. No.: US 2004/0237156 P1
(43) Pub. Date: Nov. 25, 2004

(54) Miniature rose plant named 'Meirosfon'

(75) Inventor: Alain A. Meilland, Antibes (FR)

Correspondence Address: BURNS DOANE SWECKER & MATHIS L L P, POST OFFICE BOX 1404, ALEXANDRIA, VA, 22313-1404, US

(73) Assignee: CP DELAWARE, INC., West Grove, PA

(21) Appl. No.: 10/864,386

(22) Filed: Jun. 10, 2004

Related U.S. Application Data
(63) Continuation of application No. 10/307,962, filed on Dec. 3, 2002.
Application No. 10/307,962 is a continuation of application No. 09/597,230, filed on Jun. 20, 2000, now abandoned.

(51) Int. Cl. A01H 5/00

(52) U.S. Cl. PLT/122

(57) ABSTRACT

A new and distinct variety of rose plant of the Miniature Class is provided which abundantly forms clusters of attractive double red blossoms having a light base. Commonly the blossoms only partially open even at maturity. The growth habit is bushy. Decorative medium green semi-glossy foliage is formed that contrasts nicely with the red blossoms. The plant can be readily propagated from cuttings and is particularly well suited for growing as an attractive potted gift plant.

This is a Continuation of U.S. Plant patent application Ser. No. 10/307,962, filed Dec. 3, 2002, which is a Continuation of U.S. Plant patent application Ser. No. 09/597,230, filed Jun. 20, 2000 (now abandoned).

### BOTANICAL/COMMERCIAL CLASSIFICATION

Rosa hybrida/Miniature Rose Plant

### VARIETAL DENOMINATION

cv. ‘Meirosfon’

### SUMMARY OF THE INVENTION

The new variety of Miniature rose plant was created by artificial pollination wherein two parents were crossed which previously had been studied in the hope that they would contribute the desired characteristics. The female parent (i.e., the seed parent) of the new variety was the product of the cross of the ‘Meijikatar’ variety (U.S. Plant Pat. No. 4,682) and the ‘Meilipo’ variety (U.S. Plant Pat. No. 8,504). The male parent (i.e., the pollen parent) was the ‘Meivilou’ variety (non-patented in the United States). The parentage of the new variety can be summarized as follows:

(‘Meijikatar’×‘Meilipo’)×‘Meivilou’.

The seeds resulting from the above pollination were sown and small plants were obtained which were physically and biologically different from each other. Selective study resulted in the identification of a single plant of the new variety.

It was found that the new variety of Miniature rose plant of the present invention possesses the following combination of characteristics:

(a) exhibits a bushy growth habit,

(b) forms in abundance clusters of attractive double red blossoms having a light base that commonly only partially open,

(c) forms decorative medium green semi-glossy foliage,

(d) can be readily propagated from cuttings, and

(e) is particularly well suited for growing as an attractive potted gift plant.

The profusion of red blossoms contrast nicely with the medium green foliage.

The new variety well meets the needs of the horticultural industry. It is particularly well-suited for pot forcing under greenhouse growing conditions to form an attractive gift plant.

The new variety of the present invention can be readily distinguished from its ancestors. For instance, the ‘Meijikatar’ variety forms dissimilar orange-red blossoms, the ‘Meilipo’ variety forms smaller leaves and smaller flowers, and the ‘Meivilou’ variety forms considerably longer stems.

The new variety has been found to undergo asexual propagation in France by a number of routes, including budding, grafting, and cuttage. Asexual propagation by the above-mentioned methods as performed in the South of France has shown that the characteristics of the new variety are strictly transmissible from one generation to another.

The new variety has been named the ‘Meirosfon’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were six months of age and were observed during September while growing on their own roots in greenhouses at LeCannet des Maures, Var, France. Dimensions in centimeters are shown at the bottom of the photograph.

FIG. 1—illustrates a specimen of a young shoot;

FIG. 2—illustrates specimens of two floral buds before the opening of the sepals;

FIG. 3—illustrates specimens of two floral buds at the opening of the sepals;

FIG. 4—illustrates specimens of two floral buds at the opening of the petals;

FIG. 5—illustrates specimens of two flowers during the course of opening;

FIG. 6—illustrates specimens of two open flowers—plan view—obverse;

FIG. 7—illustrates specimens of two open flowers—plan view—reverse;

FIG. 8—illustrates specimens of two fully open flowers—plan view—obverse;

FIG. 9—illustrates specimens of two fully open flowers—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates specimens of two leaves with three leaflets with the upper surface being shown at the upper left and the under surface being shown at the lower right;

FIG. 15—illustrates specimens of two leaves with five leaflets with the upper surface being shown at the lower left and the under surface being shown at the upper right; and

FIG. 16—illustrates specimens of two leaves with seven leaflets with upper surface being shown at the bottom and the under surface at the top.

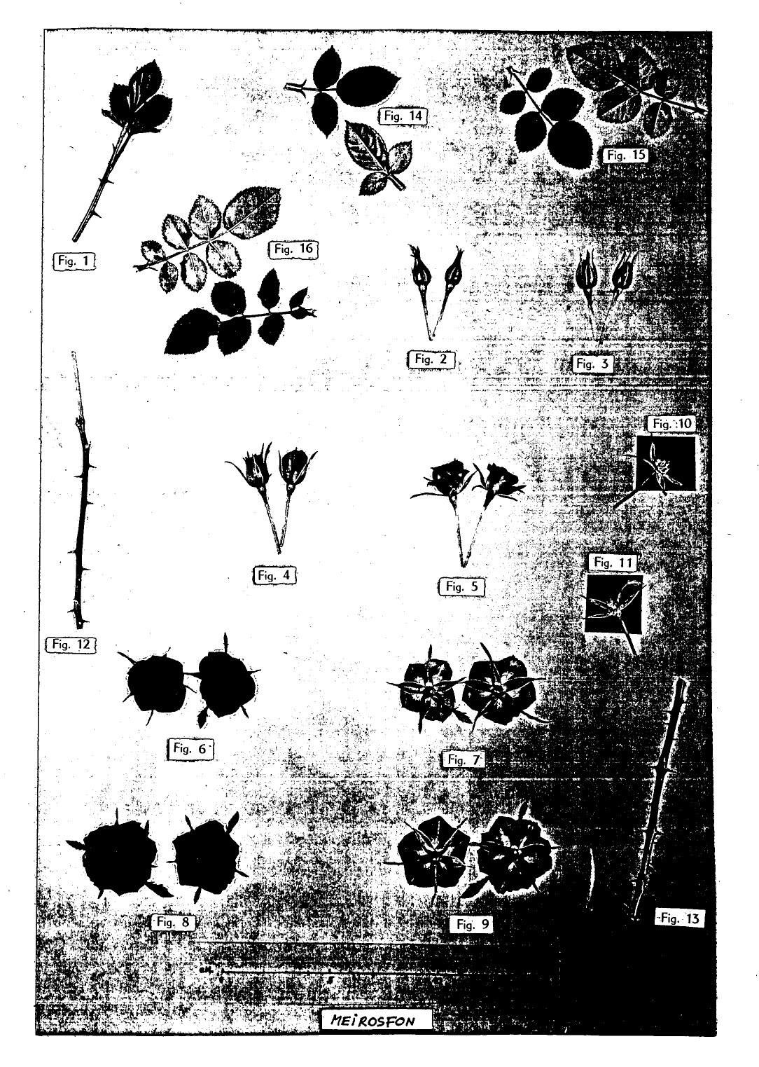

### DETAILED DESCRIPTION

The chart used in the identification of the colors is that of The Royal Horticultural Society (R.H.S. Colour Chart). The terminology which precedes reference to the chart has been added in some instances to indicate the corresponding colors in more common terms. The description is based on the observation of six month old specimens of the new variety which were observed during April while growing on their own roots in greenhouses at LeCannet des Maures, Var, France.

Class: Dwarf miniature.

Plant:

Height.—Approximately 30 to 35 cm on average at the end of the growing season.

Width.—Approximately 20 to 25 cm on average at the end of the growing season.

Habit.—Bushy.

Branches:

Color.—Young stems: medium green, Green Group 143A. — adult wood: dark green, near Green Group 137B.

Thorns.—Size: medium, and approximately 4 to 6 mm in length. — quantity: moderately sparse (as illustrated). — color: near Yellow-Green Group 153C on young stems and near Greyed-Orange Group 175C on mature wood. — configuration: fairly straight and elongated.

Leaves:

Petioles.—Upper surface: non-glandular, and near Green Group 137A in coloration. — under surface: near Green Group 138A with a few prickles.

Stipules.—Adnate, pectinate, possess a smooth surface texture, approximately 1 cm in length on average, approximately 0.4 cm in width on average, and the coloration is near Green Group 137A on the upper surface and near Green Group 138A on the under surface.

Leaflets.—Number: 3, 5, and 7 (most often). — size: the terminal leaflet is approximately 3 cm in length on average and approximately 2 cm in width on average. — shape: with a tip that is commonly acute and sometimes cuspidate and a rounded base (as illustrated). — texture: smooth. — serration: regular. — general appearance: very dense, medium green and semi-glossy. — color (young foliage): upper surface: near Green Group 137A. under surface: near Yellow-Green Group 147B. — color (adult foliage): upper surface: near Green Group 137A. under surface: near Yellow-Green Group 147B.

Inflorescence:

Number of flowers.—Commonly approximately 2 to 5 per stem.

Peduncle.—Rigid, near Yellow-Green 144A, and the length commonly is approximately 2.5 to 3 cm on average.

Sepals.—Upper surface: tomentose, and near Green Group 143B. — under surface: glabrous and near Yellow-Green Group 144A. size: elongated, commonly approximately 2 to 3 cm in length, and with some short extensions (as illustrated).

Buds.—Shape: conical. — size: medium for class, and approximately 1.5 cm in length.

Flower.—Form: double and cup-shaped. — diameter: approximately 3 cm on average when fully open. — color (when opening begins): upper surface: near Red Group 53C suffused with Red-Purple Group 57A. under surface: near Red-Purple Group 57C and at the base near Red Group 49D. — color (when blooming): upper surface: near Red Group 53A suffused with Red-Purple Group 57A. under surface: near Red-Purple Group 57C and at the base near Red Group 49D. — color (at end of opening): upper surface: near Red Group 53A suffused with Red-Purple Group 57A. under surface: near Red-Purple Group 57B and at the base near Red Group 49D. — fragrance: none. — anthers: near Yellow-Orange Group 21A in coloration and approximately 2 mm in length. — stamen number: approximately 40 to 45 on average. — filaments: yellow, approximately 4 mm in length on average, and near Green-Yellow Group 1B in coloration. — pistil number: approximately 20 to 25 on average. — petal number: approximately 30 on average. — petal texture: physically firm. — petal size: external petals commonly measure approximately 1 to 1.5 cm in length on average and approximately 0.8 to 1.9 cm in width on average, and the internal petals are shorter and commonly measure approximately 5 to 9 mm in length on average. — petal configuration: wedge-shaped base and a reflexed tip. — petaloids: none observed. — stigmas: near Yellow Group 8C. — styles: near Green-Yellow Group 1D in coloration and approximately 5 mm in length on average. — receptacle: near Yellow-Green Group 144A in coloration, smooth, approximately 5 to 6 mm in length and approximately 3 to 4 mm in width. — lasting quality: very long and approximately 10 days on the plant. — petal drop: good, the petals detach well. — hips: round, smooth, approximately 8 mm in diameter, and near Greyed-Orange Group 171C in coloration. — seeds: smooth, elongated, approximately 1 mm in length, and yellow in coloration.

Development:

Vegetation.—Strong.

Blooming.—Very abundant.

Resistance to diseases.—Very good, especially with respect to Powdery Mildew.

Aptitude to bear fruit.—Good, forms hips that are round in configuration.

I claim:

1. A new and distinct variety of Miniature rose plant characterized by the following combination of characteristics:
(a) exhibits a bushy growth habit,
(b) forms in abundance clusters of attractive double red blossoms having a light base that commonly only partially open,
(c) forms decorative medium green semi-glossy foliage,
(d) can be readily propagated from cuttings, and
(e) is particularly well suited for growing as an attractive potted gift plant;
substantially as herein shown and described.
